(19) United States
(12) Patent Application Publication
(10) Pub. No.: US 2022/0331309 A1
(43) Pub. Date: Oct. 20, 2022

(54) PHARMACEUTICAL COMPOSITION FOR TREATING INSOMNIA

(71) Applicant: Eisai R&D Management Co., Ltd., Bunkyo-ku, Tokyo (JP)

(72) Inventors: Ishani Savant Landry, Bernardsville, NJ (US); Kenya Nakai, Bunkyo-ku, Tokyo (JP); Yukiko Miyajima, Tsukuba-shi, Ibaraki (JP); Yosuke Nakatani, Tsukuba-shi, Ibaraki (JP); Takashi Ueno, Tsukuba-shi, Ibaraki (JP); Edgar Schuck, Parsippany, NJ (US)

(21) Appl. No.: 17/636,133

(22) PCT Filed: Aug. 19, 2020
(86) PCT No.: PCT/US2020/046894

§ 371 Date: Feb. 17, 2022

(30) Foreign Application Priority Data
Sep. 13, 2019 (US) PCT/US2019/051141

(51) Int. Cl. A61K 31/496; A61K 31/4709; A61K 31/70; A61P 43/00; A61K 31/135; A61K 38/00

(52) U.S. Cl. CPC A61K 31/496; A61K 31/4709; A61K 31/70; A61P 43/00; A61K 31/135; A61K 38/005

(57) ABSTRACT

The present invention provides an oral pharmaceutical composition for treating insomnia, comprising lemborexant or a pharmaceutically acceptable salt thereof, wherein a normal dose of the lemborexant or pharmaceutically acceptable salt thereof is 5 to 10 mg per day, provided that a dose of the lemborexant or pharmaceutically acceptable salt thereof is 2.5 mg per day when the pharmaceutical composition is administered to a patient together with the agent capable of moderately or strongly inhibiting CYP3A, and/or a dose of the lemborexant or pharmaceutically acceptable salt thereof is 5 mg per day when the pharmaceutical composition is administered to the patient together with the agent capable of weakly inhibiting CYP3A.

### TECHNICAL FIELD

The present invention relates to a pharmaceutical composition for treating insomnia.

### BACKGROUND ART

Two neuropeptides, Orexin-A (OX-A, a peptide consisting of 33 amino acids) and Otexin-B (OX-B, a peptide consisting of 28 amino acids), which are expressed in neurons localized in the hypothalamus of the brain, have been discovered as endogenous ligands for G protein-coupled receptors present mainly in the brain, that is, Orexin receptors (Patent Literatures 1 to 4) (Patent Literature 5, Non Patent Literature 1). It is known that the Orexin receptors include two subtypes, that is, an OX1 receptor (OX1) as a subtype 1 and an OX2 receptor (OX2) as a subtype 2. OX1 selectively binds to OX-A rather than OX-B, and OX2 binds to OX-A as well as to OX B. It has been found that Orexin stimulates food consumption of rats, suggesting a physiological function of these peptides as a mediator in the central feedback mechanism to regulate feeding behaviors (Non Patent Literature 1). On the other hand, it also has been observed that Orexin regulates the sleep-wake state; accordingly, it is considered that Orexin leads to a novel treatment method for narcolepsy as well as insomnia and other sleep disorders (Non Patent Literature 2). Furthermore, it has been suggested that Orexin signals in the ventral tegmental area in neuroplasticity associated with drug addiction and nicotine addiction play an important role in vivo (Non Patent Literature 3 and Non Patent Literature 4). It also has been reported that ethanol addiction is reduced by selectively inhibiting OX2 in an experiment using rats (Non Patent Literature 5). Furthermore, it also has been reported that in rats, a corticotropin-releasing factor (CRF) related with depression and anxiety disorder is associated with Orexin-inductive behaviors, and Orexin may play an important role in stress reactions (Non Patent Literature 6).

On the other hand, lemborexant (name of the compound: (1R,2S)-2-(((2,4-dimethylpyrimidin-5-yl)oxy)methyl)-2-(3-fluorophenyl)-N-(5-fluoropyridin-2-yl)cyclopropanecarboxamide) is known as a compound having an Orexin receptor antagonistic action and having availability as a treating agent for sleep disorders such as insomnia (Patent Literature 6).

### CITATION LIST

### Patent Literature

Patent Literature 1: WO1996/34877
Patent Literature 2: Japanese Unexamined Patent Publication No. H10-327888
Patent Literature 3: Japanese Unexamined Patent Publication No. H10-327889
Patent Literature 4: Japanese Unexamined Patent Publication No. H11-178588
Patent Literature 5: Japanese Unexamined Patent Publication No. H10-229887
Patent Literature 6: WO2016/063995

### Non Patent Literature

Non Patent Literature 1: Sakurai T. et al., Cell, 1998, 92, 573-585
Non Patent Literature 2: Chemelli R. M. et al., Cell, 1999, 98, 437-451
Non Patent Literature 3: S. L. Borgland et al., Neuron, 2006, 49.589-601
Non Patent Literature 4: C. J. Winrow et al., Neuropharmacology, 2010, 58, 185-194
Non Patent Literature 5: J. R. Shoblock et al., Psychopharmacology, 2010, 215: 191-203
Non Patent Literature 6: T. Ida et al., Biochemical and Biophysical Research Communications, 2000, 270, 318-323

### SUMMARY OF THE INVENTION

As described in Examples later, the present inventors have found a novel problem that if a pharmaceutical composition comprising lemborexant or a pharmaceutically acceptable salt thereof is administered in combination with an agent capable of inhibiting CYP3A, the concentration of lemborexant in the plasma may increase, and enhance side effects such as somnolentia. An object of the present invention is to provide a dal composition for treating insomnia which is effective and safe even if lemborexant is used in combination with an agent capable of inhibiting CYP3A.

The present invention relates to the following [1] to [32].

[1] An oral pharmaceutical composition for treating insomnia, comprising lemborexant or a pharmaceutically acceptable salt thereof, wherein a normal dose of the lemborexant or pharmaceutically acceptable salt thereof is 5 to 10 mg per day, provided that a dose of the lemborexant or pharmaceutically acceptable salt thereof is 2.5 mg per day when the pharmaceutical composition is administered to a patient together with an agent capable of moderately or strongly inhibiting CYP3A.
[2] The pharmaceutical composition according to [1], wherein the pharmaceutical composition achieves a mean AUC (0-inf) of about 113 to about 537 ng*hr/mL.
[3] The pharmaceutical composition according to [1] or [2], wherein the pharmaceutical composition achieves a mean Cmax of about 16.5 to about 56.0 ng/mL.
[4] An oral pharmaceutical composition for treating insomnia, comprising: lemborexant or a pharmaceutically acceptable salt thereof wherein a dose of the lemborexant or pharmaceutically acceptable salt thereof is 2.5 mg per day, and the pharmaceutical composition is administered to a patient together with an agent capable of moderately or strongly inhibiting CYP3A.
[5] The pharmaceutical composition according to [4], wherein the pharmaceutical composition achieves a mean AUC (0-inf) of about 308 to about 533 ng*hr/mL.
[6] The pharmaceutical composition according to [4] or [5], wherein the pharmaceutical composition achieves a mean Cmax of about 17.0 to about 26.9 ng/mL.
[7] An oral pharmaceutical composition for treating insomnia, comprising: lemborexant or a pharmaceutically acceptable salt thereof wherein a normal dose of the lemborexant or pharmaceutically acceptable salt thereof is 5 to 10 mg per day, provided that a dose of the lemborexant or pharmaceutically acceptable salt thereof is 5 mg per day when the ply composition is administered to a patient together with an agent capable of weakly inhibiting CYP3A.
[8] The pharmaceutical composition according to [7], wherein the pharmaceutical composition achieves a mean AUC (0-inf) of about 113 to about 537 ng*hr/mL.
[9] The pharmaceutical composition according to [7] or [8], wherein the pharmaceutical composition achieves a mean Cmax of about 16.5 to about 56.0 ng/mL.
[10] An oral pharmaceutical composition for treating insomnia, comprising: lemborexant or a pharmaceutically acceptable salt thereof wherein a dose of the lemborexant or pharmaceutically acceptable salt thereof is 5 mg per day, and the pharmaceutical composition is administered to a patient together with an agent capable of weakly inhibiting CYP3A.
[11] The pharmaceutical composition according to [10], wherein the pharmaceutical composition achieves a mean AUC (0-inf) of about 309 to about 337 ng*hr/mL.
[12] The pharmaceutical composition according to [10] or [11], wherein the pharmaceutical composition achieves a mean Cmax of about 16.5 to about 17.0 ng/mL.
[13] An oral pharmaceutical composition for treating insomnia, comprising: lemborexant or a pharmaceutically acceptable salt thereof wherein a normal dose of the lemborexant or pharmaceutically acceptable salt thereof is 5 to 10 mg per day, provided that a dose of the lemborexant or pharmaceutically acceptable salt thereof is 2.5 mg per day when the pharmaceutical composition is administered to a patient together with an agent capable of moderately or strongly inhibiting CYP3A, and further provided that a dose of the lemborexant or pharmaceutically acceptable salt thereof is 5 mg per day when the dal composition is administered to a patient together with an agent capable of weakly inhibiting CYP3A.
[14] An oral pharmaceutical composition for treating insomnia, comprising: lemborexant or a pharmaceutically acceptable salt thereof wherein a normal dose of the lemborexant or pharmaceutically acceptable salt thereof is 5 mg per day and is optionally increased to 10 mg per day depending on a symptom, provided that a dose of the lemborexant or pharmaceutically acceptable salt thereof is 2.5 mg per day when the pharmaceutical composition is administered to a patient together with an agent capable of moderately or strongly inhibiting CYP3A, and further provided that a dose of the lemborexant or pharmaceutically acceptable salt thereof is 5 mg per day when the pharmaceutical composition is administered to a patient together with an agent capable of weakly inhibiting CYP3A.
[15] The pharmaceutical composition according to any one of [1] to [⁶], [¹³], and [¹⁴], wherein the agent capable of moderately or strongly inhibiting CYP3A is fluconazole, erythromycin, verapamil, itraconazole, or clarithromycin.
[16] The pharmaceutical composition according to any one of [7] to [14], wherein the agent capable of weakly inhibiting CYP3A is cilostazol.
[17] A method of treating insomnia, comprising orally administering a pharmaceutical composition comprising lemborexant or a pharmaceutically acceptable salt thereof to a patient, wherein a normal dose of the lemborexant or pharmaceutically acceptable salt thereof is 5 to 10 mg per day, provided that a dose of the lemborexant or pharmaceutically acceptable salt thereof is 2.5 mg per day when the pharmaceutical composition is administered to a patient together with an agent capable of moderately or strongly inhibiting CYP3A.
[18] The method according to [17], wherein the pharmaceutical composition achieves a mean AUC (0-inf) of about 113 to about 537 ng*hr/mL.
[19] The method according to [17] or [18], wherein the pharmaceutical composition achieves a mean Cmax of about 16.5 to about 56.0 ng/mL.
[20] A method of treating insomnia, comprising orally administering a pharmaceutical composition comprising lemborexant or a pharmaceutically acceptable salt thereof to a patient, wherein a dose of the lemborexant or pharmaceutically acceptable salt thereof is 2.5 mg per day, and the pharmaceutical composition is administered to a patient together with an agent capable of moderately or strongly inhibiting CYP3A.
[21] The method according to [20], wherein the pharmaceutical composition achieves a mean AUC (0-inf) of about 308 to about 533 ng*hr/mL.
[22] The method according to [20] or [21], wherein the pharmaceutical composition achieves a mean Cmax of about 17.0 to about 26.9 ng/mL.
[23] A method of treating insomnia, comprising orally administering a pharmaceutical composition comprising lemborexant or a pharmaceutically acceptable salt thereof to a patient, wherein a normal dose of the lemborexant or pharmaceutically acceptable salt thereof is 5 to 10 mg per day, provided that a dose of the lemborexant or pharmaceutically acceptable salt thereof is 5 mg per day when the pharmaceutical composition is administered to a patient together with an agent capable of weakly inhibiting CYP3A.
[24] The method according to [23], wherein the pharmaceutical composition achieves a mean AUC (0-inf) of about 113 to about 537 ng*hr/mL.
[25] The method according to [23] or [24], wherein the pharmaceutical composition achieves a mean Cmax of about 16.5 to about 56.0 ng/mL.
[26] A method of treating insomnia, comprising orally administering a pharmaceutical composition comprising lemborexant or a pharmaceutically acceptable salt thereof to a patient, wherein a dose of the lemborexant or pharmaceutically acceptable salt thereof is 5 mg per day, and the pharmaceutical composition is administered to a patient together with an agent capable of weakly inhibiting CYP3A.
[27] The method according to [26], wherein the pharmaceutical composition achieves a mean AUC (0-inf) of about 309 to about 337 ng*hr/mL.
[28] The method according to [26] or [27], wherein the pharmaceutical composition achieves a mean Cmax of about 163 to about 17.0 ng/mL.
[29] A method of treating insomnia, comprising orally administering a pharmaceutical composition comprising lemborexant or a pharmaceutically acceptable salt thereof to a patient, wherein a normal dose of the lemborexant or pharmaceutically acceptable salt thereof is 5 to 10 mg per day, provided that a dose of the lemborexant or pharmaceutically acceptable salt thereof is 2.5 mg per day when the pharmaceutical composition is administered to a patient together with an agent capable of moderately or strongly inhibiting CYP3A, and further provided that a dose of the lemborexant or pharmaceutically acceptable salt thereof is 5 mg per day when the pharmaceutical composition is administered to a patient together with an agent capable of weakly inhibiting CYP3A.
[30] A method of treating insomnia, comprising orally administering a pharmaceutical composition comprising lemborexant or a pharmaceutically acceptable salt thereof to a patient, wherein a normal dose of the lemborexant or pharmaceutically acceptable salt thereof is 5 mg per day and is optionally increased to 10 mg per day depending on a symptom, provided that a dose of the lemborexant or pharmaceutically acceptable salt thereof is 2.5 mg per day when the pharmaceutical composition is administered to a patient together with an agent capable of moderately or strongly inhibiting CYP3A, and further provided that a dose of the lemborexant or pharmaceutically acceptable salt thereof is 5 mg per day when the pharmaceutical composition is administered to a patient together with an agent capable of weakly inhibiting CYP3A.
[31] The method according to any one of [17] to [22], [29], and [30], wherein the agent capable of moderately or strongly inhibiting CYP3A is fluconazole, erythromycin, verapamil, itraconazole, or clarithromycin.
[32] The method according to any one of [23] to [30], wherein the agent capable of weakly inhibiting CYP3A is cilostazol.

According to the present invention, a pharmaceutical composition for treating insomnia can be provided which is effective and safe even if lemborexant is used in combination with an agent capable of inhibiting CYP3A.

### BRIEF DESCRIPTION OF THE DRAWINGS

FIG. 1(a) shows the transition of the mean lemborexant concentration in the plasma from 0 to 240 hours in Test Example 3 when single doses of 1, 2.5, 5, 10, 25, 50, 100, and 200 mg lemborexant are administered to healthy adults. FIG. 1(b) shows the transition of the mean lemborexant concentration in the plasma from 0 to 24 hours in Test Example 3 when single doses of 1, 23, 5, 10, 25, 50, 100, and 200 mg lemborexant are administered to healthy adults. Each point in the graphs indicate the mean+the standard deviation.

### DESCRIPTION OF EMBODIMENTS

The content of the present invention will now be described in detail below.

In this specification, the term “lemborexant” indicates (1R,2S)-2-(((2,4-dimethylpyrimidin-5 yl)oxy)methyl)-2-(3-fluorophenyl)-N-(5-fluoropyridin-2-yl)cyclopropanecarboxamide. The structural formula is shown below.

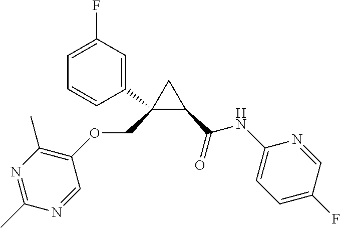 [SMILES: Cc1ncc(OC[C@@]2(c3cccc(F)c3)C[C@H]2C(=O)Nc2ccc(F)cn2)c(C)n1]

In this specification, the term “a pharmaceutically acceptable salt” is not particularly limited, and it indicates any salt which forms a salt with lemborexant, specifically, examples thereof include acid addition salts, such as inorganic acid salts, organic acid salts, or acidic amino acid salts.

Examples of one aspect of salts of inorganic acids include salts of hydrochloric acid, hydrobromic acid, sulfuric acid, nitric acid, and phosphoric acid. Examples of one aspect of salts of organic acids include salts of acetic acid, succinic acid, fumaric acid, maleic acid, tartaric acid, citric acid, lactic acid, stearic acid, benzoic acid, methanesulfonic acid, ethanesulfonic acid, and p-toluenesulfonic acid. In this specification, a dose of a pharmaceutically acceptable salt of lemborexant can be calculated based on its free form.

The lemborexant or a pharmaceutically acceptable salt thereof can be prepared by the methods described in WO2012/039371 and WO2013/123240, for example.

In this specification, the term “insomnia” indicates sleep disorders characterized by symptoms such as sleep onset insomnia, sleep maintenance insomnia, sleep offset insomnia, and nonrestorative sleep. The term “insomnia” in this specification includes transient insomnia, short-term insomnia, and long-term (chronic) insomnia.

The oral pharmaceutical composition for treating insomnia comprising lemborexant or a pharmaceutically acceptable salt thereof according to the present embodiment (hereinafter, also referred to as “pharmaceutical composition according to the present embodiment”) can be prepared by mixing the lemborexant or a pharmaceutically acceptable salt thereof with a pharmaceutically acceptable additive. The pharmaceutical composition according to the present embodiment can be prepared according to a known method such as the method described in General Rules for Preparation according to The Japanese Pharmacopoeia Sixteenth Edition, for example.

The pharmaceutical composition according to the present embodiment is orally administered to an insomnia patient, and the normal dose is 5 to 10 mg per day for an adult. The normal dose of the pharmaceutical composition according to the present embodiment can be 5 mg per day for an adult, and can be increased to 10 mg per day according to the symptom.

In this specification, the term “Cmax” indicates the maximum concentration in the plasma. The effectiveness of the lemborexant or a pharmaceutically acceptable salt thereof, particularly the action of sleep onset can be evaluated by calculating the Coax.

In this specification, the term “AUC (0-inf)” indicates the area under the plasma concentration-time curve immediately after the administration of an agent (time 0) to infinity. The effectiveness and safety of the lemborexant or a pharmaceutically acceptable salt thereof can be evaluated by calculating the AUC (0-inf).

In this specification, the temp “about” indicates numeric values within the range of ±5%.

CYP3A is one of drug-metabolizing enzymes, and is synonymous with “cytochrome P450, family 3, subfamily A”.

In this specification, the term “agent capable of moderately or strongly inhibiting CYP3A” indicates an agent which increases the AUC of CYP3A in the metabolism of a substrate twofold or more and less than fivefold (moderately inhibits CYP3A) or increases the AUC fivefold or more (strongly inhibits CYP3A) according to the classification of the CYP3A inhibiting action described in the guidances specified by the Food and Drug Administration (FDA) of the United States, specifically in Table 3-2 of “Drug Development and Drug Interactions: Table of Substrates, Inhibitors and Inducers (Nov. 14, 2017))” and Table 3 of “FDA Guidance for Industry. Drug Interaction Studies-Study Design, Data Analysis, Implications for Dosing and labeling Recommendations. Draft Guidance. February 2012”. Examples of the agent capable of moderately or strongly inhibiting CYP3A include fluconazole, erythromycin, verapamil, itraconazole, and clarithromycin.

In this specification, the term “agent capable of weakly inhibiting CYP3A” indicates an agent which increases the AUC of CYP3A in the metabolism of a substrate 1.25-fold or more and less than twofold according to the guidances of the FDA. Example of the agent capable of weakly inhibiting CYP3A includes cilostazol.

When the pharmaceutical composition according to the present embodiment is administered to the patient together with the agent capable of moderately or strongly inhibiting CYP3A, the dose of the lemborexant or pharmaceutically acceptable salt thereof is 2.5 mg per day. With this dose of the lemborexant or pharmaceutically acceptable salt thereof; the effectiveness of the pharmaceutical composition according to the present embodiment could be compatible with the safety thereof.

When the normal dose of the lemborexant or pharmaceutically acceptable salt thereof is 5 to 10 mg per day in the pharmaceutical composition according to the present embodiment, provided that the dose of the lemborexant or pharmaceutically acceptable salt thereof is 2.5 mg per day if the pharmaceutical composition is administered to the patient together with the agent capable of moderately or strongly inhibiting CYP3A, the pharmaceutical composition achieves the mean AUC (0-inf) of about 113 to about 537 ng*hr/mL in one aspect.

When the normal dose of the lemborexant or pharmaceutically acceptable salt thereof is 5 to 10 mg per day in the pharmaceutical composition according to the present embodiment, provided that the dose of the lemborexant or pharmaceutically acceptable salt thereof is 2.5 mg per day if the pharmaceutical composition is administered to the patient together with the agent capable of moderately or strongly inhibiting CYP3A, the pharmaceutical composition achieves a mean Cmax of about 16.5 to about 56.0 ng/mL in one aspect.

When the pharmaceutical composition according to the present embodiment is administered to the patient together with the agent capable of moderately or strongly inhibiting CYP3A and the dose of the lemborexant or pharmaceutically acceptable salt thereof is 2.5 mg per day, the pharmaceutical composition achieves a mean AUC (0-inf) of about 308 to about 533 ng*hr/mL in one aspect, achieves a mean AUC (0-inf) of about 308 to about 445 ng*hr/mL in another aspect, and achieves a mean AUC (0-inf) of about 374 to about 533 ng*hr/mL in further another aspect. If the mean AUC (0-inf) is within the above range, the effectiveness and safety of the pharmaceutical composition according to the present embodiment used in combination with the agent capable of moderately or strongly inhibiting CYP3A could be secured. Here, the team “mean AUC (0-inf)” indicates the geometric mean of the AUC (0-inf).

When the pharmaceutical composition according to the present embodiment is administered to the patient together with the agent capable of moderately or strongly inhibiting CYP3A and the dose of the lemborexant or pharmaceutically acceptable salt thereof is 2.5 mg per day, the pharmaceutical composition achieves a mean Cmax of about 17.0 to about 26.9 ng/mL, achieves a mean Cmax of about 17.0 to about 21.1 ng/mL in one aspect, and achieves a mean Cmax of about 18.1 to about 26.9 ng/mL in another aspect. If the mean Cmax is within the above range, the effectiveness (particularly, action of sleep onset) of the pharmaceutical composition according to the present embodiment used in combination with the agent capable of moderately or strongly inhibiting CYP3A could be secured. Here, the term “mean Cmax” indicates the geometric mean of the Cmax.

When the pharmaceutical composition according to the present embodiment is administered to the patient together with the agent capable of weakly inhibiting CYP3A, the dose of the lemborexant or pharmaceutically acceptable salt thereof is 5 mg per day. With this dose of the lemborexant or pharmaceutically acceptable salt thereof the effectiveness of the pharmaceutical composition according to the present embodiment could be compatible with the safety.

When the normal dose of the lemborexant or pharmaceutically acceptable salt thereof is 5 to 10 mg per day in the pharmaceutical composition according to the present embodiment, provided that the dose of the lemborexant or pharmaceutically acceptable salt thereof is 5 mg per day if the pharmaceutical composition is administered to the patient together with the agent capable of weakly inhibiting CYP3A, the pharmaceutical composition achieves a mean AUC (0-inf) of about 113 to about 537 ng*hr/mL in one aspect.

When the normal dose of the lemborexant or pharmaceutically acceptable salt thereof is 5 to 10 mg per day in the pharmaceutical composition according to the present embodiment, provided that the dose of the lemborexant or pharmaceutically acceptable salt thereof is 5 mg per day if the pharmaceutical composition is administered to the patient together with the agent capable of weakly inhibiting CYP3A, the pharmaceutical composition achieves a mean Cmax of about 16.5 to about 56.0 ng/mL in one aspect.

When the pharmaceutical composition according to the present embodiment is administered to the patient together with the agent capable of weakly inhibiting CYP3A and the dose of the lemborexant or pharmaceutically acceptable salt thereof is 5 mg per day, the pharmaceutical composition achieves a mean AUC (0-inf) of about 309 to about 337 ng*hr/mL in one aspect. If the mean AUC (0-inf) is within the above range, the effectiveness and safety of the pharmaceutical composition according to the present embodiment used in combination with the agent capable of weakly inhibiting CYP3A could be secured.

When the pharmaceutical composition according to the present embodiment is administered to the patient together with the agent capable of weakly inhibiting CYP3A and the dose of the lemborexant or pharmaceutically acceptable salt thereof is 5 mg per day, the pharmaceutical composition achieves a mean Cmax of about 16.5 to about 17.0 ng/mL in one aspect. If the mean Cmax is within the above range, the effectiveness (particularly, action of sleep onset) of the pharmaceutical composition according to the present embodiment used in combination with the agent capable of weakly inhibiting CYP3A could be secured.

### EXAMPLES

The following examples illustrate various aspects of the present invention and are not to be interpreted as limiting the scope of the invention.

### [Test Example 1 ] Influences of Agent Capable of Moderately or Strongly Inhibiting CYP3A on Pharmacokinetics of Lemborexant in Healthy Adults

(1-1: Administration of lemborexant in combination with fluconazole (agent capable of moderately inhibiting CYP3A))

A single dose of 10 mg lemborexant was administered to fourteen healthy adults (males and females, 18 to 55 years old) (single administration of lemborexant); 200 mg of fluconazole was administered one time per day (two times only on Day 11) from Day 11 to Day 26 where the single administration of the lemborexant was defined as Day 1; and a single dose of 10 mg lemborexant was administered on Day 15 (administration of lemborexant in combination with fluconazole). The concentrations of lemborexant in the plasma during the single administration of lemborexant and during the administration of lemborexant in combination with fluconazole were measured with a high performance liquid chromatograph/tandem mass spectrometry (hereinafter, referred to as “LC-MS/MS”) under the following conditions to calculate the geometric means of the Cmax and the AUC (0-inf). The results are shown in Table 1.

### (Apparatuses Used)

HPLC (pump: LC-10ADvp or LC 20AD, Autosampler SLL 20ACHT, SHIMADZU Corporation) mass spectrometer (API5000 or API5500, AB Sciex)

### (Pre-Treatment)

A 10 μL solution of an internal standard substance (lemborexant labeled with deuterium) was added to 100 μL of a human plasma, and the resulting mixture was convened into a basic condition with 10 μL of aqueous ammonia and was subjected to liquid-liquid extraction with methyl tert-butyl ether (MTBE). After stirring (for about 10 minutes) and centrifugation (about 14000 rpm, for about 10 minutes) were performed, the upper layer was evaporated into dryness at 30° C. under a nitrogen stream, and was redissolved with 200 μL of a mixed solution (50/50, v/v) of 0.1% formic acid-containing acetonitrile/water to prepare a sample for LC-MS/MS.

### (Conditions for Measurement)

mobile phase A: 0.1% formic acid aqueous solution
mobile phase B: acetonitrile
analysis column: Phenomenex Kinetic XB-C18 (5 μm, 4.6×250 mm)
time for measurement 17.5 minutes
gradient condition: The mobile phase B was maintained at 35% from 0 to 2 minutes, was linearly increased to 38% from 2 minutes to 5 minutes, was maintained at 38% from 5 minutes to 11.5 minutes, was linearly increased to 55% from 11.5 minutes to 12.1 minutes, was maintained at 55% from 12.1 minutes to 14.5 minutes, was decreased to 35% at 14.6 minutes, and was maintained to 17.5 minutes.
flow rate: 12 mL/min from 0 to 7 minutes, decelerated to 0.7 mL/min from 7 minutes to 8 minutes, maintained at 0.7 mL/min from 8 minutes to 11.6 minutes, accelerated to 12 mL/min from 11.6 minutes to 12 minutes, and maintained at 12 mL/min to 17.5 minutes. The measurement was performed in an MRM mode by positive detection of an electrospray ionization (ESI) method. The MRM transition (combination of a precursor ion and a product ion) of lemborexant used was m/z 411>287 (CE30). The internal standard substance used was lemborexant labeled with deuterium, and m/z 414>290 (CE18) was used. A variety of parameters for AP15500/5500 Qtrap used are shown below. Ion spray Voltage: 5500 v, Curtain Gas 40, CAD 8, Gas1 70, Gas2 70, DP100, Dwell time 250 (parameters are not limited to these)

The lemborexant concentration in the plasma was calculated using an internal standard calibration curve created through inverse regression according to the method of least squares generated from the ratio of the peak area of lemborexant to that of the internal standard substance.

(1-2: Administration of Lemborexant in Combination with Itraconazole (Agent Capable of Strongly Inhibiting CYP3A))

A single dose of 10 mg lemborexant was administered to fifteen healthy adults (males and females, 21 to 55 years old) (single administration of lemborexant); 200 mg of itraconazole was administered one time per day from Day 15 to Day 34 where the single administration of lemborexant was defined as Day 1; and a single dose of 10 mg lemborexant was administered on Day 22 (administration of lemborexant in combination with itraconazole). The lemborexant concentrations in the plasma during the single administration of lemborexant and during the administration of lemborexant in combination with itraconazole were measured by LC-MS/MS under the same condition as that in (1-1) of Test Example 1, and the geometric mean s of the Cmax and the AUC (0-inf) were calculated. The results are shown in Table 2.

TABLE 1

| | | Administration of | |
|---|---|---|---|
| | Single | lemborexant in | |
| | administration of | combination with | Ratio of |
| | lemborexant (a) | fluconazole (b) | (b) to (a) |
| Cmax (ng/mL) | 54.4 | 88.3 | 1.62 |
| AUC(0-inf) | 374 | 1480 | 4.17 |
| (ng*hr/mL) | | | |

TABLE 2

| | | Administration of | |
|---|---|---|---|
| | Single | lemborexant in | |
| | administration of | combination with | Ratio of |
| | lemborexant (c) | itraconazole (d) | (d) to (c) |
| Cmax (ng/mL) | 54.3 | 73.9 | 1.36 |
| AUC(0-inf) | 433 | 1580 | 3.70 |
| (ng*hr/mL) | | | |

From Table 1, in the administration of lemborexant in combination with fluconazole, the mean Cmax of lemborexant 62% increased and the mean AUC (0-inf) 317% increased, respectively, compared to the single administration of lemborexant. From Table 2, in the administration of lemborexant in combination with itraconazole, the mean Cmax of lemborexant 36% increased and the mean AUC (0-inf) 270% increased, respectively, compared to the single administration of lemborexant. This suggested that if lemborexant was administered in combination with an agent capable of inhibiting CYP3A, the lemborexant concentration in the plasma might increase and it might enhance the side effects such as somnolentia.

(1-3) Prediction of Pharmacokinetics of Lemborexant During Administration of Lemborexant in Combination with Agent Capable of Moderately or Strongly Inhibiting CYP3A

Because the Cmax and the AUC (0-inf) of lemborexant are proportional to the dose of lemborexant, based on the mean Cmax and the mean AUC (0-inf) of lemborexant during the administration of 10 mg lemborexant in combination with fluconazole and during the administration of 10 mg lemborexant in combination with itraconazole, which were obtained in Sections (1-1) and (1-2) above, predicted values of the mean Cmax and the mean AUC (0-inf) of lemborexant (both were geometric means) during administration of 2.5 mg lemborexant in combination with fluconazole and during administration of 2.5 mg lemborexant in combination with itraconazole were calculated. The results are shown in Tables 3 and 4 with the actually measured values of the single administration of 10 mg lemborexant in Sections (1-1) and (1-2) above.

TABLE 3

| Single administration of | | Administration of lemborexant (2.5 mg) | |
|---|---|---|---|
| lemborexant (10 mg) | | in combination with fluconazole | |
| | AUC(0-inf) | | AUC(0-inf) |
| Cmax (ng/mL) | (ng*hr/mL) | Cmax (ng/mL) | (ng*hr/mL) |
| 54.4 | 374 | 22.1 | 370 |
| (46.7, 63.3) | (314, 446) | (18.1, 26.9) | (308, 445) |

* The numerals within the brackets indicate the lower limit value and the upper limit value of the 95% confidence interval, respectively.

TABLE 4

| Single administration of | | Administration of lemborexant (2.5 mg) | |
|---|---|---|---|
| lemborexant (10 mg) | | in combination with itraconazole | |
| | AUC(0-inf) | | AUC(0-inf) |
| Cmax (ng/mL) | (ng*hr/mL) | Cmax (ng/mL) | (ng*hr/mL) |
| 54.3 | 433 | 18.9 | 447 |
| (47.4, 62.3) | (349, 537) | (17.0, 21.1) | (374, 533) |

* The numerals within the brackets indicate the lower limit value and the upper limit value of the 95% confidence interval, respectively.

It was verified that the predicted values of the mean Cmax and mean AUC (0-inf) of lemborexant when 2.5 mg lemborexant was administered in combination with fluconazole were 59.4% and 1.1% lower than the mean Cmax and mean AUC (0-inf) of lemborexant when a single dose of 10 mg lemborexant was administered, respectively. It was also verified that for the predicted values of the mean Cmax and mean AUC (0-inf) of lemborexant when 2.5 mg lemborexant was administered in combination with itraconazole, the mean Cmax was 652% lower and the mean AUC (0-inf) was 32% higher than the mean Cmax and mean AUC (0-inf) of lemborexant when a single dose of 10 mg lemborexant was administered. It should be noted that the upper limit value of the 95% confidence interval of the predicted value of the mean AUC (0-inf) of lemborexant when 2.5 mg lemborexant was administered in combination with itraconazole was lower than the upper limit value of the 95% confidence interval of the mean AUC (0-inf) of lemborexant when a single dose of 10 mg lemborexant was administered.

Accordingly, the predicted value of the mean AUC (0-inf) of lemborexant when 2.5 mg lemborexant was used in combination with the agent capable of moderately or strongly inhibiting CYP3A approximated to the mean AUC (0-inf) of lemborexant when a single dose of 10 mg lemborexant was administered. Accordingly, it is considered that the effectiveness and the safety when 2.5 mg lemborexant is used in combination with the agent capable of moderately or strongly inhibiting CYP3A are equal to those when a single dose of 10 mg lemborexant is administered.

Moreover, the predicted value of the mean Cmax of lemborexant when 2.5 mg lemborexant was used in combination with the agent capable of moderately or strongly inhibiting CYP3A was lower than the mean Cmax of lemborexant when a single dose of 10 mg lemborexant was administered while the predicted value approximated to the mean Cmax (22.3 ng/mL) of lemborexant when a single dose of 5 mg lemborexant was administered to six healthy adults (males and females, 32 to 53 years old). Accordingly, it is considered that the effectiveness (particularly, action of sleep onset) when 2.5 mg lemborexant was used in combination with the agent capable of moderately or strongly inhibiting CYP3A was equal to that when a single dose of 5 mg lemborexant was administered.

From these, it is recommended that the dose of lemborexant used in combination with the agent capable of moderately or strongly inhibiting CYP3A is 2.5 mg per day. From Tables 3 and 4, it was also verified that when 2.5 mg lemborexant was used in combination with the agent capable of moderately or strongly inhibiting CYP3A, lemborexant achieved a mean AUC (0-inf) of about 308 to about 533 ng*hr/mL (the lower limit value of the 95% confidence interval in use in combination with fluconazole to the upper limit value of the 95% confidence interval in use in combination with itraconazole), a mean AUC (0-inf) of about 308 to about 445 ng*hr/mL (the lower limit value of the 95% confidence interval in use in combination with fluconazole to the upper limit value of the 95% confidence interval in use in combination with fluconazole), or a mean AUC (0-inf) of about 374 to about 533 ng*hr/mL (the lower limit value of the 95% confidence interval in use in combination with itraconazole to the upper limit value of the 95% confidence interval in use in combination with itraconazole). Furthermore, from Tables 3 and 4, it was verified that when 2.5 mg lemborexant was used in combination with the agent capable of moderately or strongly inhibiting CYP3A, lemborexant achieved a mean Cmax of about 17.0 to about 26.9 ng/mL (the lower limit value of the 95% confidence interval in use in combination with itraconazole to the upper limit value of the 95% confidence interval in use in combination with fluconazole), a mean Cmax of about 17.0 to about 21.1 ng/mL (the lower limit value of the 95% confidence interval in use in combination with itraconazole to the upper limit value of the 95% confidence interval in use in combination with itraconazole), or a mean Cmax of about 18.1 to about 26.9 ng/mL (the lower limit value of the 95% confidence interval in use in combination with fluconazole to the upper limit value of the 95% confidence interval in use in combination with fluconazole).

### [Test Example 2] Influences of Agent Capable of Weakly Inhibiting CYP3A on Pharmacokinetic of Lemborexant Using Physiologically-Based Pharmacokinetic (PBPK) Model

A PBPK model for lemborexant was constructed using a Simcyp (registered trademark) simulator (Jamei, 2009) to predict the drug interaction when lemborexant was administered in combination with fluoxetine (agent capable of weakly inhibiting CYP3A). Specifically, the following condition was set for the prediction of drug interaction, and Simcyp (registered trademark) was used to predict the AUC (0-inf) and the Cmax when a single close of 10 mg lemborexant was administered; then, the AUC (0-inf) and the Cmax when a single dose of 5 mg lemborexant was administered were calculated considering that the AUC (0-inf) and the Cmax of lemborexant were proportional to the dose of lemborexant (Table 5). Furthermore, based on the calculated values above, the AUC ratio and Cmax ratio (both were geometric mean s) of lemborexant administered in combination with fluoxetine to lemborexant during single administration of lemborexant were calculated. Predicted influences on drug interaction were also evaluated based on the guidance of drug interaction specified by the Food and Drug Administration (FDA) of the United States. The results are shown in Table 6.

(Setting Condition in Administration of Lemborexant in Combination with Fluoxetine)
Target of administration: 100 Sim-Healthy Volunteers (males and females, 20 to 50 years old)
lemborexant: a single dose of 10 mg was administered on Day 25 from the start of the test.
fluoxetine: a dose of 40 mg was administered one time per day from Day 1 to Day 39 from the start of the test.

The drug interaction when lemborexant was administered in combination with erythromycin, verapamil, or fluvoxamine (each is an agent capable of moderately inhibiting CYP3A) was also predicted by the same method as above. The results are shown in Table 6.

(Setting Condition in Administration of Lemborexant in Combination with Erythromycin, Verapamil, or Fluvoxamine)
Target of administration: 100 Sim-Healthy Volunteers (males and females, 20 to 50 years old)
lemborexant a single dose of 10 mg was administered on Day 8 from the start of the test.
erythromycin: a dose of 500 mg was administered every six hours from Day 1 to Day 20 from the start of the test.
verapamil: a dose of 80 mg was administered three times per day from Day 1 to Day 20 from the start of the test.
fluvoxamine: a dose of 50 mg was administered one time per day from Day 1 to Day 20 from the start of the test.

TABLE 5

| | | |
|---|---|---|
| Geometric | Lower limit of 90% | Upper limit of 90% |
| mean | confidence interval | confidence interval |

During single administration of 10 mg lemborexant

| | | | |
|---|---|---|---|
| Cmax (ng/mL) | 27.68 | 26.17 | 29.29 |
| AUC(0-inf) | 366.04 | 335.61 | 399.23 |
| (ng*hr/mL) | | | |

During single administration of 5 mg lemborexant

| | |
|---|---|
| Cmax (ng/mL) | 13.84 |
| AUC(0-inf) | 183.02 |
| (ng*hr/mL) | |

TABLE 6

| | Classification of CYP3A | | | |
|---|---|---|---|---|
| Drug used in | inhibiting action of drug | | | Predicted influences |
| combination | used in combination | AUC ratio | Cmax ratio | on drug interaction |
| Fluoxetine | Weak | 1.77 (1.69-1.84) | 1.21 (1.19-1.23) | Weak |
| Erythromycin | Moderate | 4.33 (4.03-4.66) | 1.46 (1.42-1.50) | Moderate |
| Verapamil | Moderate | 3.87 (3.59-4.17) | 1.43 (1.40-1.46) | Moderate |
| Fluvoxamine | Moderate | 1.09 (1.08-1.09) | 1.06 (1.06-1.07) | No influences |

* The numerals within the brackets indicate the 90% confidence interval.

From Table 6, it was suggested that fluoxetine weakly affects lemborexant, erythromycin and verapamil moderately affect lemborexant, and fluvoxamine does not affect lemborexant, and it was verified that the classifications of the CYP3A inhibiting actions of the drugs used in combination were similar to the predicted influences on the interaction. In other words, it was inferred that the pharmacokinetics of the lemborexant is weakly affected by use in combination with fluoxetine.

From these, it is recommended that the dose of lemborexant used in combination with the agent capable of weakly inhibiting CYP3A is 5 mg per day. A mean AUC (0-inf) of about 309 to about 337 ng*hr/mL is calculated by multiplying the AUC (0-inf) (Table 5 above) when a single dose of 5 mg lemborexant is administered by the lower limit value of the 90% confidence interval of the AUC ratio of the agent capable of weakly inhibiting CYP3A (fluoxetine) and the AUC (0-inf) by the upper limit value (Table 6 above), respectively, and a mean Cmax of about 16.5 to about 17.0 ng/mL is calculated by multiplying the Cmax (Table 5 above) when a single dose of 5 mg lemborexant is administered by the lower limit value of the 90% confidence interval of the Cmax ratio of the agent capable of weakly inhibiting CYP3A (fluoxetine) and the Cmax by the upper limit value (Table 6 above), respectively. Accordingly, it was verified that if 5 mg lemborexant is used in combination with the agent capable of weakly inhibiting CYP3A, lemborexant achieves a mean AUC (0-inf) of about 309 to about 337 ng*hr/mL and a mean Cmax of about 16.5 to about 17.0 ng/mL.

### [Test Example 3] Single Dose Administration Test Targeting Healthy Adults and Primary Insomnia (KEYNOTE-001 Study, Part A)

In 64 cases of healthy adults under a feed-deprived condition, the pharmacokinetics of lemborexant administered with single doses of 1, 2.5, 5, 10, 25, 50, 100, and 200 mg thereof was examined by a randomized, double-blinded, placebo-controlled, multi-stage single administration test. In each group, lemborexant was administered to six cases, and the placebo was administered to two cases.

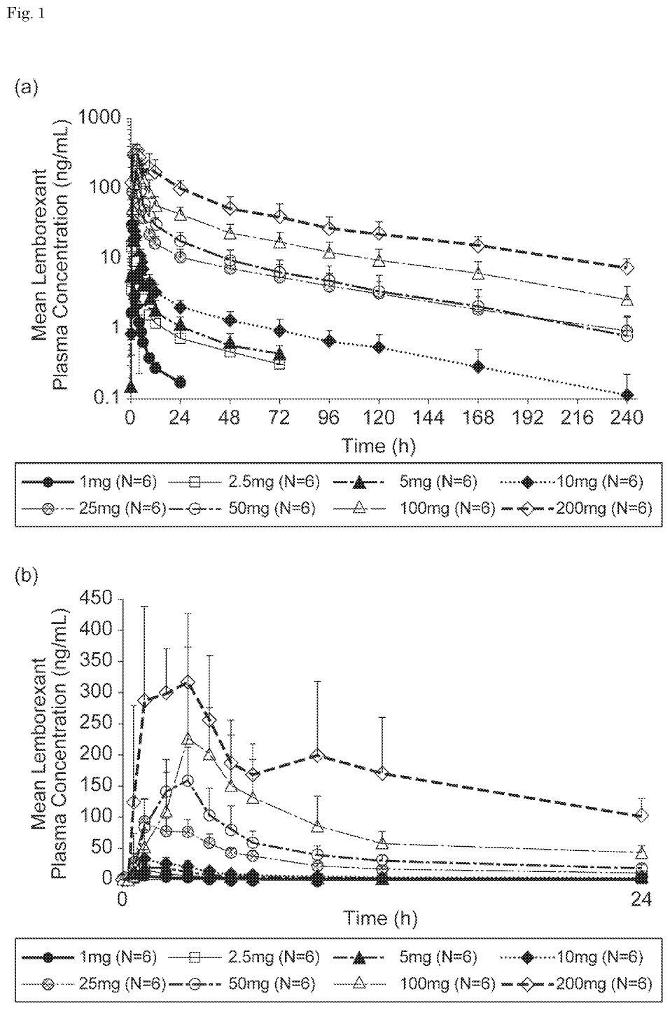

The transition of the mean lemborexant concentration in the plasma when single doses of 1, 25, 5, 10, 25, 50, 100, and 200 mg lemborexant were administered to the healthy adults is shown in FIG. 1. The pharmacokinetic parameters when single doses of 5 mg and 10 mg lemborexant were administered are shown in Table 7.

The lemborexant concentration in the plasma after the administration exhibited biphasic elimination. The median of the tmax after the administration of 1, 2.5, 5, and 10 mg lemborexant was 1 to 155 bouts, and the median of the tmax after the administration with a dose of 25 mg or more was 2 to 3 hocus. Although the geometric mean of the Cmax of lemborexant was increased with an increase in dose, the geometric mean of the Cmax for a dose of 10 mg or more was increased with a proportion slightly lower than the dose ratio. The mean of the AUC (0-24 h) exhibited approximately dose proportionality in the examined dose range. It is believed that in all the administration groups, the exposure until 9 hours after the administration, which is believed to reflect pharmacological action associated with treatment of insomnia, was about 75%, in average, of the exposure until 24 hours after the administration and was about 45% of the AUC (0-inf). The lemborexant concentration in the plasma after 9 hours from administration of a single dose of 2.5 to 10 mg lemborexant was about 10 to 13% of the Cmax.

The relations between the lemborexant concentration in the plasma and pharmacodynamic evaluations (Digit Symbol Substitution Test (DSST), Psychomotor Vigilance Task (PVT), and Karolinska Sleepiness Scale (KSS)) were examined; as a result, although the correlation was barely found in the dose up to 5 mg, a correlation was found in the dose of 10 mg or more between the lemborexant concentration in the plasma and DSST, PVT, and PVT.

TABLE 7

| | | Single | | Single | |
|---|---|---|---|---|---|
| | | administration | | administration | |
| | | of 5 mg | | of 10 mg | |
| | | lemborexant | | lemborexant | |
| Cmax | The number of cases | 6 | | 6 | |
| (ng/mL) | Mean (SD) | 22.7 | (4.39) | 36.0 | (18.7) |
| | Median | 21.9 | | 31.4 | |
| | Minimum value, | 17.2, | 30.0 | 16.9, | 62.1 |
| | maximum value | | | | |
| | Geometric mean | 22.3 | (19.1) | 32.0 | (57.3) |
| | (% CV) | | | | |
| AUC(0-inf) | The number of cases | 5 | | 6 | |
| (ng*hr/mL) | Mean (SD) | 149 | (34.3) | 311 | (90.1) |
| | Median | 139 | | 314 | |
| | Minimum value, | 123, | 209 | 188, | 409 |
| | maximum value | | | | |
| | Geometric mean | 146 | (20.9) | 299 | (31.7) |
| | (% CV) | | | | |

The lower limit value and upper limit value of the 95% confidence interval of the Cmax (ng/mL) when a single dose of 5 mg or 10 mg lemborexant was administered are as shown in Table 8.

TABLE 8

| Dose of | Lower limit | Upper limit |
|---|---|---|
| administration | value | value |
| 5 mg | 18.3 | 27.3 |
| 10 mg | 18.3 | 56.0 |

The lower limit value and upper limit value of the 95% confidence interval of the AUC (0-inf) (ng*hr/mL) when a single dose of 5 mg or 10 mg lemborexant was administered are as shown in Table 9.

TABLE 9

| Dose of | Lower limit | Upper limit |
|---|---|---|
| administration | value | value |
| 5 mg | 113 | 189 |
| 10 mg | 216 | 414 |

From above, it was verified that when the dose of lemborexant is 5 to 10 mg, lemborexant achieves a mean AUC (0-inf) of about 113 to about 537 ng*hr/mL (the lower limit value of the 95% confidence interval in Table 9 during administration of a single of 5 mg lemborexant to the upper limit value of the 95% confidence interval in Table 4 during administration of a single dose of 10 mg lemborexant). It was also verified that when the dose of lemborexant was 5 to 10 mg, lemborexant achieves a mean Cmax of about 16.5 to about 56.0 ng/mL (the lower limit value in use of 5 mg lemborexant in combination with the agent capable of weakly inhibiting CYP3A to the upper limit value of the 95% confidence interval in Table 8 during administration of a single dose of 10 mg lemborexant).

1. An oral pharmaceutical composition for treating insomnia, comprising:
lemborexant or a pharmaceutically acceptable salt thereof,
wherein a normal dose of the lemborexant or pharmaceutically acceptable salt thereof is 5 to 10 mg per day, provided that a dose of the lemborexant or pharmaceutically acceptable salt thereof is 2.5 mg per day when the pharmaceutical composition is administered to a patient together with an agent capable of moderately or strongly inhibiting CYP3A.

2. The pharmaceutical composition according to claim 1, wherein the pharmaceutical composition achieves a mean AUC (0-inf) of 113 to 537 ng*hr/mL.

3. The pharmaceutical composition according to claim 1, wherein the pharmaceutical composition achieves a mean Cmax of 16.5 to 56.0 ng/mL.

4. An oral pharmaceutical composition for treating insomnia, comprising:
lemborexant or a pharmaceutically acceptable salt thereof,
wherein a dose of the lemborexant or pharmaceutically acceptable salt thereof is 2.5 mg per day, and the pharmaceutical composition is administered to a patient together with an agent capable of moderately or strongly inhibiting CYP3A.

5. The pharmaceutical composition according to claim 4, wherein the pharmaceutical composition achieves a mean AUC (0-inf) of 308 to 533 ng*hr/mL.

6. The pharmaceutical composition according to claim 4, wherein the pharmaceutical composition achieves a mean Cmax of 17.0 to 26.9 ng/mL.

7. An oral pharmaceutical composition for treating insomnia, comprising:
lemborexant or a pharmaceutically acceptable salt thereof,
wherein a normal dose of the lemborexant or pharmaceutically acceptable salt thereof is 5 to 10 mg per day, provided that a dose of the lemborexant or pharmaceutically acceptable salt thereof is 5 mg per day when the pharmaceutical composition is administered to a patient together with an agent capable of weakly inhibiting CYP3A.

8. The pharmaceutical composition according to claim 7, wherein the pharmaceutical composition achieves a mean AUC (0-inf) of 113 to 537 ng*hr/mL.

9. The pharmaceutical composition according to claim 7, wherein the pharmaceutical composition achieves a mean Cmax of 16.5 to 56.0 ng/mL.

10. An oral pharmaceutical composition for treating insomnia, comprising:
lemborexant or a pharmaceutically acceptable salt thereof,
wherein a dose of the lemborexant or pharmaceutically acceptable salt thereof is 5 mg per day, and the pharmaceutical composition is administered to a patient together with an agent capable of weakly inhibiting CYP3A.

11. The pharmaceutical composition according to claim 10, wherein the pharmaceutical composition achieves a mean AUC (0-inf) of 309 to 337 ng*hr/mL.

12. The pharmaceutical composition according to claim 10, wherein the pharmaceutical composition achieves a mean Cmax of 16.5 to 17.0 ng/mL.

13. An oral pharmaceutical composition for treating insomnia, comprising:
lemborexant or a pharmaceutically acceptable salt thereof,
wherein a normal dose of the lemborexant or pharmaceutically acceptable salt thereof is 5 to 10 mg per day, provided that a dose of the lemborexant or pharmaceutically acceptable salt thereof is 2.5 mg per day when the pharmaceutical composition is administered to a patient together with an agent capable of moderately or strongly inhibiting CYP3A, and further provided that a dose of the lemborexant or pharmaceutically acceptable salt thereof is 5 mg per day when the pharmaceutical composition is administered to a patient together with an agent capable of weakly inhibiting CYP3A.

14. An oral pharmaceutical composition for treating insomnia, comprising:
lemborexant or a pharmaceutically acceptable salt thereof,
wherein a normal dose of the lemborexant or pharmaceutically acceptable salt thereof is 5 mg per day and is optionally increased to 10 mg per day depending on a symptom, provided that a dose of the lemborexant or pharmaceutically acceptable salt thereof is 2.5 mg per day when the pharmaceutical composition is administered to a patient together with an agent capable of moderately or strongly inhibiting CYP3A, and further provided that a dose of the lemborexant or pharmaceutically acceptable salt thereof is 5 mg per day when the pharmaceutical composition is administered to a patient together with an agent capable of weakly inhibiting CYP3A.

15. The pharmaceutical composition according to claim 1, wherein the agent capable of moderately or strongly inhibiting CYP3A is fluconazole, erythromycin, verapamil, itraconazole, or clarithromycin.

16. The pharmaceutical composition according to claim 7, wherein the agent capable of weakly inhibiting CYP3A is cilostazol.

17. A method of treating insomnia, comprising orally administering a pharmaceutical composition comprising lemborexant or a pharmaceutically acceptable salt thereof to a patient, wherein a normal dose of the lemborexant or pharmaceutically acceptable salt thereof is 5 to 10 mg per day, provided that a dose of the lemborexant or pharmaceutically acceptable salt thereof is 2.5 mg per day when the pharmaceutical composition is administered to a patient together with an agent capable of moderately or strongly inhibiting CYP3A.

18. The method according to claim 17, wherein the pharmaceutical composition achieves a mean AUC (0-inf) of about 113 to about 537 ng*hr/mL.

19. The method according to claim 17, wherein the pharmaceutical composition achieves a mean Cmax of about 16.5 to about 56.0 ng/mL.

20. A method of treating insomnia, comprising orally administering a pharmaceutical composition comprising lemborexant or a pharmaceutically acceptable salt thereof to a patient, wherein a dose of the lemborexant or pharmaceutically acceptable salt thereof is 2.5 mg per day, and the pharmaceutical composition is administered to a patient together with an agent capable of moderately or strongly inhibiting CYP3A.

21. The method according to claim 20, wherein the pharmaceutical composition achieves a mean AUC (0-inf) of about 308 to about 533 ng*hr/mL.

22. The method according to claim 20, wherein the pharmaceutical composition achieves a mean Cmax of about 17.0 to about 26.9 ng/mL.

23. A method of treating insomnia, comprising orally administering a pharmaceutical composition comprising lemborexant or a pharmaceutically acceptable salt thereof to a patient, wherein a normal dose of the lemborexant or pharmaceutically acceptable salt thereof is 5 to 10 mg per day, provided that a dose of the lemborexant or pharmaceutically acceptable salt thereof is 5 mg per day when the pharmaceutical composition is administered to a patient together with an agent capable of weakly inhibiting CYP3A.

24. The method according to claim 23, wherein the pharmaceutical composition achieves a mean AUC (0-inf) of about 113 to about 537 ng*hr/mL.

25. The method according to claim 23, wherein the pharmaceutical composition achieves a mean Cmax of about 16.5 to about 56.0 ng/mL.

26. A method of treating insomnia, comprising orally administering a pharmaceutical composition comprising lemborexant or a pharmaceutically acceptable salt thereof to a patient, wherein a dose of the lemborexant or pharmaceutically acceptable salt thereof is 5 mg per day, and the pharmaceutical composition is administered to a patient together with an agent capable of weakly inhibiting CYP3A.

27. The method according to claim 26, wherein the pharmaceutical composition achieves a mean AUC (0-inf) of about 309 to about 337 ng*hr/mL.

28. The method according to claim 26, wherein the pharmaceutical composition achieves a mean Cmax of about 16.5 to about 17.0 ng/mL.

29. A method of treating insomnia, comprising orally administering a pharmaceutical composition comprising lemborexant or a pharmaceutically acceptable salt thereof to a patient, wherein a normal dose of the lemborexant or pharmaceutically acceptable salt thereof is 5 to 10 mg per day, provided that a dose of the lemborexant or pharmaceutically acceptable salt thereof is 2.5 mg per day when the pharmaceutical composition is administered to a patient together with an agent capable of moderately or strongly inhibiting CYP3A, and further provided that a dose of the lemborexant or pharmaceutically acceptable salt thereof is 5 mg per day when the pharmaceutical composition is administered to a patient together with an agent capable of weakly inhibiting CYP3A.

30. A method of treating insomnia, comprising orally administering a pharmaceutical composition comprising lemborexant or a pharmaceutically acceptable salt thereof to a patient, wherein a normal dose of the lemborexant or pharmaceutically acceptable salt thereof is 5 mg per day and is optionally increased to 10 mg per day depending on a symptom, provided that a dose of the lemborexant or pharmaceutically acceptable salt thereof is 2.5 mg per day when the pharmaceutical composition is administered to a patient together with an agent capable of moderately or strongly inhibiting CYP3A, and further provided that a dose of the lemborexant or pharmaceutically acceptable salt thereof is 5 mg per day when the pharmaceutical composition is administered to a patient together with an agent capable of weakly inhibiting CYP3A.

31. The method according to claim 17, wherein the agent capable of moderately or strongly inhibiting CYP3A is fluconazole, erythromycin, verapamil, itraconazole, or clarithromycin.

32. The method according to claim 23, wherein the agent capable of weakly inhibiting CYP3A is cilostazol.
